(19) United States
(12) Patent Application Publication
(10) Pub. No.: US 2009/0053707 A1
(43) Pub. Date: Feb. 26, 2009

(54) Methods for lightening skin and hair

(76) Inventors: Mina Yaar, Sharon, MA (US); Hee-Young Park, Weston, MA (US); Vladimir Botchkarev, Sharon, MA (US); Barbara A. Gilchrest, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/983,207

(22) Filed: Nov. 7, 2007

Related U.S. Application Data
(63) Continuation of application No. 10/963,432, filed on Oct. 12, 2004, now abandoned.
Application No. 10/963,432 is a continuation-in-part of application No. PCT/US03/11376, filed on Apr. 11, 2003.
(60) Provisional application No. 60/372,523, filed on Apr. 12, 2002.

(51) Int. Cl. C12Q 1/68; C12Q 1/02; C12Q 1/48; G01N 33/573

(52) U.S. Cl. 435/6; 435/29; 435/15; 435/7.4

(57) ABSTRACT

Methods are described wherein the skin of a vertebrate, or the skin or hair of a mammal can be lightened by administration of an agent, e.g., protein, peptide, active fusion protein, active fragment, or molecular mimic, that binds to BMP-4 transmembrane receptors on melanocytes and decreases the level of melanin synthesis. Also described are methods to identify molecules that mimic the function of BMP-4 in causing a decrease in melanin in melanocytes.

### RELATED APPLICATION(S)

This application is a continuation of U.S. application Ser. No. 10/963,432, filed Oct. 12, 2004, which is a continuation-in-part of International Application No. PCT/US03/11376, which designated the United States and was filed Apr. 11, 2003, published in English, which claims the benefit of U.S. Provisional Application No. 60/372,523, filed Apr. 12, 2002. The entire teachings of the above applications are incorporated herein by reference.

### BACKGROUND OF THE INVENTION

Currently, areas of unwanted cutaneous hyperpigmentation are treated with “bleaching” agents. The same preparations are used to lighten normal skin color in persons of darker complexion who wish to appear more fair-skinned. The most common active ingredient in these preparations is hydroquinone, an intrinsically toxic chemical that inhibits melanization by a poorly understood mechanism. At the most effective concentrations, hydroquinone may lead to permanent melanocyte loss. Other “active” ingredients in available bleaching creams, such as kojic acid, have the same problems of minimal efficacy and potential toxicity. Hair is conventionally lightened by “bleaching,” a harsh chemical process that damages the hair shaft while reducing its pigment content. As well, “bleaching” treats only that portion of the hair above the skin surface, so that darker “roots” are soon visible, detracting from the desired cosmetic effect.

U.S. Pat. No. 5,962,417 describes methods to decrease constitutive and induced cutaneous pigmentation by inhibiting PKC-β. However, in these methods the active molecule must enter the cell, the site of PKC-β, which is anticipated to be more problematic than a method in which an agent binds to a transmembrane receptor.

### SUMMARY OF THE INVENTION

The present invention is based on Applicants' discovery that bone morphogenetic protein 4 (BMP-4) decreases melanin synthesis in melanocytes by decreasing the activity of tyrosinase, the rate limiting enzyme in melanogenesis.

The invention comprises a method of decreasing pigmentation in the skin or hair in a mammal, by administration of an effective amount of a composition of BMP-4, an active fusion protein of BMP-4, an active fragment of BMP-4, or a combination of the foregoing. In one embodiment of the invention, the mammal receiving the treatment is a human. In another embodiment of the invention, the BMP-4, active fusion protein of BMP-4, active fragment of BMP-4 or combination reduces the level of tyrosinase in epidermal melanocytes, thus decreasing pigmentation. In other embodiments of the invention, the BMP-4 composition is administered topically. The composition can be formulated in liposomes or as an aerosol or as any other cosmetically acceptable carrier such as a cream, ointment, gel, lotion or solution.

The invention further comprises a method of decreasing pigmentation in epidermal melanocytes in a vertebrate, by application of an effective amount of a composition comprising BMP-4, an active fragment of BMP-4, an active fusion protein of BMP-4, or a combination of the foregoing. The method can result in a decrease in pigment by a reduction in the level of tyrosinase in the melanocytes. In one embodiment of the invention, the composition is in the form of liposomes.

Also provided is a method of decreasing pigmentation in the skin of a mammal, comprising the step of administering to the mammal an effective amount of BMP-4 or BMP-4 fusion protein, active fragment of BMP-4, molecular mimic of BMP-4 including the membrane receptor binding site on BMP-4, or a combination of the foregoing.

Part of the invention consists of methods to determine which agents or chemical compounds act as mimics of BMP-4 in its ability to inhibit melanin synthesis in melanocytes. Fragments of BMP-4 or mimics of BMP-4 or other chemical compounds that could potentially act on melanocytes in the same manner as BMP-4 can be tested for their effects on cells. For many of these methods, the cells best suited are cells of melanocytic lineage, defined here as melanocytes and melanoma cells. For assays wherein melanin production is measured, if a melanoma cell line is to be used, it is best to select a cell line that produces a high level of melanin, as levels of melanin are not readily measurable in some melanomas.

In one method of the invention, mimics or fragments of BMP-4 that inhibit melanin synthesis in cells of melanocytic lineage are identified by culturing such cells in the presence of the fragment or mimic. The melanin content in the cells is then measured and compared to the melanin content measured in control cells not cultured in the presence of the fragment or mimic. A lower melanin content in the cells treated with the mimic or fragment of BMP-4 than the melanin content in the control cells is indicative that the fragment or mimic inhibits melanin synthesis in cells of melanocytic lineage.

In another embodiment of the invention, mimics or fragments of BMP-4 that inhibit melanin synthesis in melanocytes are identified by adding the fragment or mimic to cells of melanocytic lineage in culture, measuring the change in melanin content in the cells after addition of the fragment or mimic, and comparing the melanin content to the melanin content measured in control cells not cultured in the presence of the fragment or mimic. A lower melanin content in the cells treated with the mimic or fragment of BMP-4 as compared to the melanin content in the control cells is indicative that the fragment or mimic inhibits synthesis in melanocytes. In a particular embodiment of the invention, the cells to be used to identify BMP-4 active fragments or mimics are melanocytes isolated from newborn humans.

The present invention comprises another method of identifying an agent that mimics the activity of BMP-4 in melanocytes. The method involves incubating cells of melanocytic lineage with the agent in culture and assaying for nuclear factor kappa B

(NF-κB) in the nuclei. More nuclei showing NF-κB staining in the cells incubated with the agent as compared to the number of nuclei showing NF-κB staining in control cells not incubated with the agent in culture is indicative that the agent mimics the activity of BMP-4 in melanocytes.

A further embodiment of the present invention relates to another method of identifying an agent that mimics the activity of BMP-4 on melanocytes. Cells of melanocytic lineage are transfected with DNA, as in a vector, comprising a BMP-4 responsive promoter operably linked to a reporter gene. The cells are then exposed to the agent to be tested. In this embodiment of the invention, the amount of gene product produced is determined for both the transfected culture exposed to the agent and for a control transfected culture with no exposure to the agent. A greater or lower amount of gene product produced as a result of reporter gene expression in the culture of transfected cells contacted with the agent as compared to a control culture of transfected cells not contacted with the agent is indicative that the test agent mimics the activity of BMP-4 on melanocytes.

In another embodiment of the present invention, methods are provided for identifying a BMP-4 fragment or mimic which decreases the level of melanin in melanocytes by affecting the level of tyrosinase in the cells. The BMP-4 fragment or mimic is first incubated with cells of melanocytic lineage in culture. The level of tyrosinase mRNA is then determined in the cells and compared to the level of tyrosinase mRNA isolated from control cells not incubated with the BMP-4 fragment or mimic. If the level of tyrosinase mRNA determined in the cultures exposed to the BMP-4 fragment or mimic is lower than the level of tyrosinase mRNA determined in the control melanocytes, then the BMP-4 fragment or mimic decreases the level of melanin in melanocytes.

One aspect of the invention relates to a method for identifying a BMP-4 fragment or mimic which decreases the level of melanin in melanocytes, by incubating the BMP-4 fragment or mimic with cells of melanocytic lineage in culture and then determining the level of PKC-β RNA in the cells. This level is compared to the level of PKC-β RNA determined in control cells not incubated with the BMP-4 fragment or mimic. If the level of PKC-β RNA determined in the cells incubated with the BMP-4 mimic is lower than the level of PKC-β RNA determined in the control cells, then the BMP-4 fragment or mimic decreases the level of melanin in melanocytes.

An additional aspect of the invention relates to identifying a BMP-4 fragment or mimic which decreases the level of melanin in melanocytes, by incubating the BMP-4 fragment or mimic with cells of melanocytic lineage in culture and then determining the level of PKC-β protein in the cells. The level of PKC-β protein determined to be in the cells is compared with a level of PKC-β protein determined to be in control cells not incubated with the BMP-4 fragment or mimic. If the level of PKC-β protein in the treated cells is lower than the level of PKC-β protein determined in the control cells, then the BMP-4 fragment or mimic decreases the level of melanin in melanocytes.

A further aspect of the invention for identifying a BMP-4 fragment or mimic which decreases the level of melanin in melanocytes comprises incubating the BMP-4 fragment or mimic with cells of melanocytic lineage in culture, staining the cells with anti-NF-κB antibodies and comparing the distribution of antibody staining of these cells with the distribution of antibody staining of control cells not incubated with the BMP-4 fragment or mimic. If the distribution of the antibody staining of the cells incubated with the BMP-4 fragment or mimic is predominantly nuclear compared to predominantly cytoplasmic and perinuclear in the control cells, then the BMP-4 fragment or mimic decreases the level of melanin in melanocytes.

Another embodiment of the present invention relates to an additional method for identifying a BMP-4 fragment or mimic which decreases the level of melanin in melanocytes. The BMP-4 fragment or mimic is first incubated with cells of melanocytic lineage in culture. Northern blot analysis is then performed on total cellular RNA isolated from the cells at various time intervals of incubation of the BMP-4 fragment or mimic with the cells, using DNA encoding human tyrosinase as a probe. The northern blot analysis performed on the cells incubated with BMP-4 fragment or mimic is compared to the northern blot analysis performed on total cellular RNA isolated from control cells not incubated with the BMP-4 fragment or mimic. If the level of tyrosinase-specific RNA seen in the treated cells decreases over time of incubation with the BMP-4 fragment or mimic, compared to the level of tyrosinase-specific RNA seen for the control cells, then the BMP-4 fragment or mimic decreases the level of melanin in melanocytes.

Yet another method of the invention for identifying a BMP-4 fragment or mimic which decreases the level of melanin in melanocytes comprises incubating a putative BMP-4 fragment or mimic with cells of melanocytic lineage in culture. Northern blot analysis of total cellular RNA isolated from these cells at various time intervals of incubation of the BMP-4 fragment or mimic with these cells is then performed, using DNA encoding human PKC-β as a probe, thereby quantifying PKC-β RNA. The results of the northern blot analysis on the cells incubated with the BMP-4 fragment or mimic is compared with results of northern blot analysis performed on total cellular RNA isolated from control cells not incubated with the BMP-4 fragment or mimic. If the level of PKC-β-specific RNA seen in the cells incubated with the BMP-4 fragment or mimic decreases over time, compared to the level of PKC-β-specific RNA seen for the control cells, then the BMP-4 fragment or mimic decreases the level of melanin in melanocytes.

Also provided is a method for identifying a BMP-4 fragment or mimic which decreases the level of melanin in melanocytes, comprising adding the BMP-4 fragment or mimic to cells of melanocytic lineage in culture and producing an extract of total cellular proteins from samples of the cells taken at several times after addition of the BMP-4 fragment or mimic. These isolated proteins are separated by gel electrophoresis, blotted on a membrane for western blot analysis, and the membrane is incubated with anti-tyrosinase antibodies, thereby allowing the binding of a saturating amount of antibodies to the tyrosinase. A means to detect the bound antibodies is applied to the membrane, thereby quantifying bound antibodies. If saturating amounts of bound antibodies decrease with time after addition of the BMP-4 fragment or mimic to the cells of melanocytic lineage, then the BMP-4 fragment or mimic decreases the level of melanin in melanocytes.

In a further embodiment, the invention relates to a method of altering the regulation of melanogenesis during the hair cycle in the hair, wool or fur of a mammal, comprising administering to the mammal an effective amount of a composition comprising BMP-4, an active fusion protein of BMP-4, an active fragment of BMP-4, a BMP mimic or a combination of the foregoing. Such alteration in regulation of melanogeneis can result in decreasing pigmentation of the hair, wool or fur of the mammal.

The invention also comprises a method of decreasing pigmentation in the skin or hair in a mammal by administering to the mammal an effective amount of a composition comprising an agent that activates the BMP type 2 and type 1 receptors on the surface of melanocytes or melanoma cells.

In yet a further embodiment, the invention relates to a method of switching pigment production from eumelanin to pheomelanin in the follicular melanocytes of a mammal, said method comprising administering to the mammal an effective amount of a composition comprising BMP-4, an active fusion protein of BMP-4, an active fragment of BMP-4, a BMP mimic or a combination of the foregoing.

The invention also comprises assays to find agents that mimic BMP-4 in their effect on decreasing melanin production in cells of melanocytic lineage. BMP-4 also has other effects on cells with the appropriate receptors for BMP-4. These effects can be assayed and used to predict the effect of an agent on melanin production in cells of melanocytic lineage.

In one method for determining the effect of an agent on melanin production in cells, the steps include: contacting the cells in culture with the agent, wherein the cells produce tyrosinase and produce BMP receptor type 1A, BMP receptor type 1B and BMP receptor type 2; measuring the amount of tyrosinase in the cells contacted with the agent; and measuring the amount of tyrosinase in control cells. If the amount of tyrosinase per cell contacted with the agent is less than the amount of tyrosinase per cell measured in the control cells, then the agent decreases melanin production in the cells. If the amount of tyrosinase per cell measured in the cells contacted with the agent is greater than the amount of tyrosinase per cell in the control cells, then the agent increases melanin production in the cells.

In another method for determining the effect of an agent on melanin production in cells, the cells produce BMP receptor type 1A, BMP receptor type 1B and BMP receptor type 2. The steps include contacting cells in culture with the agent, measuring the amount of microphthalmia-associated transcription factor in the cells contacted with the agent, and measuring the amount of microphthalmia-associated transcription factor in control cells. If the amount of microphthalmia-associated transcription factor per cell measured in the cells contacted with the agent is less than the amount of microphthalmia-associated transcription factor per cell measured in the control cells, then the agent decreases melanin production in the cells. If the amount of microphthalmia-associated transcription factor per cell measured in the cells contacted with the agent is greater than the amount of microphthalmia-associated transcription factor per cell measured in the control cells, then the agent increases melanin production in the cells.

In another method to identify an agent that reduces melanin production in cells of melanocytic lineage, the cells comprise in their cell membranes functional BMP receptor type 1A, BMP receptor type 1B and BMP receptor type 2, and the cells further comprise one or more genetic contructs comprising a BMP-4 responsive promoter operably linked to a reporter gene, with the agent. The method includes the steps of contacting the cells, thereby producing one or more cultures of cells contacted with the agent, and measuring the amount of gene product produced as a result of reporter gene expression in one or more cultures of cells contacted with the agent and in one or more cultures of control cells. A greater or lesser amount of gene product produced as a result of reporter gene expression in one or more cultures of cells contacted with the agent, compared to the amount of gene product in one or more cultures of control cells, is indicative that the agent being tested reduces melanin production in melanin-producing cells. In particular embodiments, the BMP-4 responsive promoter can be the tyrosinase promoter or the microphthalmia-associated transcription factor promoter. A lesser amount of gene product produced as a result of reporter gene expression in one or more cultures of cells contacted with the agent, compared to the amount of gene product in one or more cultures of control cells, is indicative that said agent reduces melanin production in melanin-producing cells.

In a different method of identifying an agent that reduces melanin production in melanin-producing cells, the method requires starting with cells that have in their cell membranes functional BMP receptor type 1A, BMP receptor type 1B and BMP receptor type 2, and at least the steps of transfecting cells with DNA, as in a vector, comprising a BMP-4 responsive promoter operably linked to a reporter gene, thereby producing transfected cells, contacting the transfected cells with the agent, thereby producing one or more cultures of transfected cells contacted with the agent, and measuring the amount of gene product produced as a result of reporter gene expression in the cultures of transfected cells contacted with the agent and in one or more cultures of control cells. A greater or lesser amount of gene product produced as a result of reporter gene expression in one or more cultures of transfected cells contacted with the agent compared to the amount of gene product in one or more cultures of control cells is indicative that the agent increases or reduces, respectively, melanin production in cells of melanocytic lineage. In particular embodiments, the BMP-4 responsive promoter can be the tyrosinase promoter or the microphthalmia-associated transcription factor promoter, and a lesser amount of gene product produced as a result of reporter gene expression in one or more cultures of transfected cells contacted with the agent compared to the amount of gene product in one or more cultures of control cells is indicative that said agent reduces melanin production in melanin-producing cells.

### BRIEF DESCRIPTION OF THE DRAWINGS

FIG. 1 is a bar graph plotting melanin (pg/cell) produced after the addition of 0, 0.5, 1, 2, 4, 5, 10 or 25 ng/ml BMP-2/4 to cultures of human newborn melanocytes. Cells were processed for the melanin assay 72 hours after stimulation.

FIG. 2 is a bar graph of cell yield v. BMP-2/4 (ng/ml).

FIG. 3A is a bar graph plotting ELISA units indicating by-products of apoptosis 48 hours after treatment of cultures of melanocytes with diluent, BMP-2/4, UV radiation or DNA histone complex included as a positive control.

FIG. 3B is a bar graph plotting ELISA units indicating by-products of apoptosis 72 hours after treatment of cultures of melanocytes with diluent, BMP-2/4, UV radiation or DNA histone complex.

FIG. 4 represents a nucleotide sequence (SEQ ID NO: 1), which is part of the tyrosinase promoter and which contains the sequence (CAGACA) recognized for BMP-4 signaling.

FIG. 5 is a bar graph depicting CAT activity 72 hours after AN melanoma cells were transfected with a tyrosinase-CAT plasmid construct and treated with BMP-2/4 or a diluent.

### DETAILED DESCRIPTION OF THE INVENTION

The present invention is based on Applicants' finding that bone morphogenetic protein 4 (BMP-4) decreases pigmentation in human melanocytes. BMP-4 acts by reducing the production of melanin in these cells.

Melanocytes (pigment cells) are cells located in the basal layer of the epidermis and in the hair bulb. Melanin pigment is deposited in melanocyte-specific organelles called melanosomes that are then transferred from the melanocyte to surrounding keratinocytes so that pigment is widely dispersed through the epidermis (outer layer) of the skin or the hair shaft. The color (pigmentation) of vertebrate skin, and the skin, hair, wool and fur of mammals is determined largely by its melanin pigment content.

Bone morphogenetic proteins (BMPs), members of the TGF-β superfamily, were originally identified and characterized by their ability to induce cartilage and bone formation during embryogenesis and morphogenesis. Since their initial discovery, BMPs were found to participate in cellular processes in the mature as well as the developing organism. BMPs are highly conserved across vertebrate and mammalian species (Hwang, S. L. et al., DNA Cell Biol. 16(8):1003-1011 (1997); Martinez-Barbera et al., Gene 198(1-2):33-59 (1997); Hwang, S. L. et al., Biochem. Biophys. Res. Comm. 258:(2):457-463 (1999); Panopoulou et al., Dev. Dyn. 213(1):130-139 (1998)). BMPs also play a role in several non-osteogenic processes including directing the development of neural crest cells (Jin et al., Develop. Bio., 233(1):22-37 (2001)). Of the more than 20 BMPs isolated to date, six are structurally related to each other and are capable of initiating the process of endochondrial bone formation (Subach, B. R. et al., Neurosurg. Focus 10(4):1-6 (2001)). Human BMP-2 and human BMP-4 have both been sequenced and characterized (Kawai, S. and Sugiura, T. Bone 29(1):54-61 (2001); Padgett, R. W. et al., Proc. Natl. Acad. Sci. 90:2905 (1993); Wozney et al., Science 248(4855):1528-1534 (1988)). There is homology between BMP-2 and BMP-4 that in some parts of the gene (base 3242-3468 of BMP-2 and base 37-263 of BMP-4) is 100%. See http//www.nci.nlm.nih.gov/entrez, see also Kawai and Sugiura, 2001; Wozney et al., 1988. BMP-4 has been shown to contribute to somite development by inhibiting myogenesis (McPherron, A. C. and S-J Lee, Proc. Natl. Acad. Of Sci., USA 94:12457 (1997)), while BMP-2 induces the formation of chondryocyte and osteoblast precursors (Wall, N. A. and Hogan, B. L. M., Curr. Opin. Genet. Dev. 4:517 (1994)).

The invention encompasses a preparation to decrease pigmentation of skin or hair, comprising a disulfide linked homodimeric protein, herein exemplified by a fusion protein abbreviated BMP-2/4. The DNA sequence encoding the human BMP-2 signal peptide and propeptide (amino acid residues 1-282 of human BMP-2) has been fused to the human BMP-4 mature chain (amino acid residues 293-408 of human BMP-4) (Wozney et al., 1988). The fusion protein was expressed in the mouse melanoma cell line NSO (Wozney, et al., 1998). The mature BMP-2/4 fusion protein was generated by the proteolytic removal of the signal peptide and propeptide (Hammonds, Jr. et al., U.S. Pat. No. 5,168,050; Hammonds, et al., Mol. Endocr. 5(1):149-155 (1991)). The molecular weight of BMP-2/4 is 26 kD. Smaller fragments of this molecule, peptide or non-peptide analogs having activity similar to that of BMP-4 can be identified.

Wozney et al., (1998), discloses the cDNAs for BMP-2 and BMP-4 cloned from human placental DNA and encoding the full length BMP-2 and BMP-4 proteins. BMP-2 and BMP-4 share over 50% amino acid sequence homology, with particularly high homology at the 3′ carboxy terminal end (92% similarity) (Wozney et al., 1998). The BMP-2 and BMP-4 DNA sequences were first cloned into expression plasmids. Then, an expression plasmid containing DNA encoding the N-terminal prodomain of BMP-2 spliced to the C-terminal mature growth factor domain of the BMP-4 was constructed. The recombinant BMP-2/4 protein showed the ability to increase calcium content in rat bone implants (U.S. Pat. No. 5,168,050). Applicants' research shows that the BMP-2/4 fusion protein has the biological activity of BMP-4.

The bone morphogenetic proteins, including BMP-4, signal through two transmembrane receptors, BMP receptor 1 and BMP receptor 2 (Piek et al., FASEBJ 13(15):2105-24 (1999), Massague et al., Genes and Develop. 14:627-644 (2000)). BMP receptor 2 is the major ligand binding receptor, while BMP receptor 1 primarily transduces the signal into the cell (Piek et al., 1999; Massague et al., 2000). There are two distinct forms of BMP receptor 1: A and B (Piek et al., 1999; Massague et al., 2000). Upon ligand binding, type 1 and type 2 receptors form heteromeric complexes (Piek et al., 1999; Massague et al., 2000). Thus both type 1 and type 2 receptors are thought to be required to mediate BMP signaling. Upon binding to its receptors, BMP-4 has been reported to stimulate the activation of NF-κB (Mohan R. R. et al., Invest. Ophthalmol. Vis. Sci. 39:2626 (1998)). In its inactive state NF-κB is present in the cytoplasm but rapidly translocates to the nucleus upon activation.

RT-PCR using primers complementary to these human receptor transcripts, confirmed by product sequencing, clearly demonstrated strong constitutive expression of the proliferation-associated BMP receptor type 1A (BMP R1A) in melanocytes. See Example 7. Melanocytes also expressed BMP receptor type 1B (BMP R1B) and BMP receptor type 2 (BMP R2), demonstrating that melanocytes express all transmembrane receptors required to respond to BMP stimulation. See Examples 8 and 9. Additionally, melanoma cells also express BMP receptor type 1A, receptor type 1B and receptor type 2, and can also be used to examine BMP-4 effects, for example, in methods to identify a BMP-4 mimic or active fragment of BMP-4. See Examples 11 and 12. BMP signals are transduced through phosphorylation of cytoplasmic Smad proteins that translocate to the nucleus to effect gene transcription. The DNA consensus sequence recognized by Smad, CAGACA, is present in the promoter region of human tyrosinase (nucleotides -2051 to -2045). Northern and western blot analyses of BMP-4-treated melanocytes show substantially downregulated tyrosinase mRNA and protein, respectively, within 15 hours through 72 hours. See Examples 3 and 4. BMP-4 also substantially downregulated the two PKC-β mRNA transcripts as well as PKC-β protein, the enzyme that activates tyrosinase. See Examples 5 and 6. RT-PCR experiments and sequencing of the products revealed that keratinocytes weakly express BMP-4, but melanocytes strongly express BMP-4, suggesting an autocrine and paracrine effect of BMP-4 on melanocytes. These data provide evidence of a novel signaling pathway that influences melanocyte function in human skin.

Activation of tyrosinase, the rate-limiting enzyme in melanogenesis, results from the protein kinase C-beta (PKC-β) mediated phosphorylation of serine and threonine residues of the cytoplasmic domain of tyrosinase (U.S. Pat. No. 5,962,417). Tyrosinase, found exclusively in melanocytes, is a transmembrane protein localized to the melanosomes. Inhibition of the PKC-β-mediated phosphorylation of tyrosinase prevents activation of tyrosinase in epidermal melanocytes, which results in a decrease in the production of melanin pigment in melanocytes. A decrease in the level of tyrosinase or PKC-β therefore results in a decrease in the level of melanin produced in the cells.

Herein is described a method for decreasing pigmentation in the skin or hair in a mammal, wherein the method comprises administering to the mammal an effective amount of a composition comprising an agent that activates BMP type 2 and BMP type 1 receptors, by their phosphorylation (Piek et al., 1999; Massague et al., 2000), on the surface of melanocytes or melanoma cells. One end result of the affected pathways in these cells is a decrease in melanin content per cell. Other measurable effects include decreases in tyrosinase mRNA and protein, decreases in PKC-β mRNA and protein, decreases in microphthalmia-associated transcription factor (MITF) mRNA and protein, migration of NF-κB to the nuclei, and phosphorylation of the BMP type 2 and type 1 receptors. Any agent activating the BMP type 2 and type 1 receptors, causing their phosphorylation, can be used to decrease melanin content in melanocytes in skin. Such agents can include, for example, BMP-4, BMP-4 fusion proteins, BMP-4 fragments and molecular mimics of BMP-4 which phosphorylate the BMP type 2 and type 1 receptors. Other BMPs, fusion proteins, fragments and molecular mimics derived from them can also be found which activate BMP type 2 and type 1 receptors by phosphorylation, and which ultimately decrease melanin content per melanocyte in skin. Other intermediate effects as described above can also be observed upon activation of the BMP receptors with other BMPs and molecules derived from them.

To identify agents that decrease pigmentation in the skin or hair of a mammal, one can test for agents that activate the BMP type 2 receptor and BMP type 1A and 1B receptors by phosphorylation. This can be done using melanocytes or melanoma cells, or other cells demonstrated to have BMP type 1A and 1B and BMP type 2 receptors.

Provided are methods of decreasing the level of pigmentation in mammalian skin or hair by application of BMP-4, an active fusion protein of BMP-4, an active fragment of BMP-4, or a combination of the foregoing. These methods comprise contacting mammalian epidermal cells in such a manner that the BMP-4 fragment or mimic enters the cells, including basal layer melanocytes (e.g., introducing into, delivering to, or administering to), with an effective amount of a BMP-4 fragment or mimic which decreases or depresses pigmentation. For example, the BMP-4 fragment or mimic can be contained in a physiologically compatible composition which is topically applied to the skin, or the skin surrounding hair, wool or fur bulbs, or both, so that both skin and hair can be lightened.

BMP-4 to be used in the methods of the invention can be purified from natural sources, or can be isolated from cells or cell cultures, optionally using cells comprising genomes altered by recombinant methods. The BMP-4 can have the amino acid sequence of any naturally occurring allelic variant, or can be a mutant form resulting from an artificial genetic manipulation resulting in single or multiple amino acid substitutions, deletions or insertions, or some combination of genetic alterations. Truncated BMP-4 proteins, and fragments of any of the above-described BMP-4 proteins which can be shown to have the qualitative biological activity of naturally occurring BMP-4 or the BMP-2/4 fusion protein described herein, are also included as “BMP-4” and “active BMP-4 fragments.”

As used herein, the term “fusion protein” refers to a fusion, through a peptide bond of (1) one protein molecule or a portion thereof to (2) another protein molecule, a portion thereof (for example, a domain), an amino acid residue, or a peptide, the amino acid sequence of which may or may not be derived from a naturally-occurring protein. Part (2) of the fusion protein can be N-terminal, C-terminal, or internal to part (1). The fusion proteins of the present invention can include constructs in which a linker peptide sequence is utilized. Fusion proteins can have a formula of R₁-R₂ or R₁-L-R₂, where R₁ and R₂ are substantially similar or identical protein molecules and L is a linker, typically a peptide. R₁ and R₂ can be different proteins, for example.

As used herein, the term “biologically active fragment” or “active fragment” encompasses fragments (e.g., portions and peptides) of BMP-4 which are capable of exhibiting the same qualitative biological activity of BMP-4, although the quantitative activity may be different from that of BMP-4. These molecules are capable of binding to the BMP-4 receptors expressed by melanin-producing cells. A “fragment” of a protein is any polypeptide or peptide having a contiguous segment of the amino acid sequence of the protein which is shorter than the protein. Such molecules may or may not also comprise additional amino acid residues not naturally occurring in BMP-4, for example, those derived from a process of cloning or from linker peptides.

The invention arises from the discovery that BMP-4 inhibits PKC-β-mediated phosphorylation of tyrosinase and prevents activation of tyrosinase in epidermal melanocytes. This results in a decrease in the production of melanin pigment in melanocytes.

The precise dose to be employed in the formulation of a composition to decrease pigmentation of the skin or hair will depend on the route of administration and the desired effect. An effective amount of such an identified BMP-4 fragment or mimic is an amount effective to measurably decrease, reduce or substantially inhibit pigmentation in epidermal melanocytes. The concentration of melanin in melanocytes can be evaluated using the methods described herein, or other methods known in the art.

Various delivery systems suitable for use in the present invention are known to those of skill in the art and can be used to deliver effective amounts of BMP-4 fragment or mimic, or fusion protein such as BMP-2/4, to decrease pigmentation in melanocytes. In general, any formulation that can penetrate the skin barrier (stratum corneum) is preferred. For example, encapsulation in liposomes, microparticles, or microcapsules; expression by recombinant cells, receptor-mediated endocytosis, construction of a naturally-occurring or pseudo-ligand encoding nucleic acid as part of a retroviral or other vector can be used. In addition, the compositions of the invention may be formulated in various solvents, gels, creams, lotions or solutions to facilitate simple application to the skin and/or hair follicles. Aerosolized compositions, comprising a suspension of very fine particles of a solid or droplets of a liquid in a gaseous medium, may also be utilized to deliver effective amounts of the BMP-4, BMP-4 fusion protein, fragment or mimic. The suspension is stored under high pressure and released in the form of a fine spray or foam and can be applied directly to the skin or hair.

In a preferred embodiment, a liposome preparation can be used. The liposome preparation can be comprised of any liposome which penetrates the stratum corneum and fuses with the cell membrane, resulting in delivery of the contents of the liposome into the cell. Liposomes can be prepared by methods well-known to those of skill in the art. For example, liposomes such as those described in U.S. Pat. No. 5,077,211; U.S. Pat. No. 4,621,103; U.S. Pat. No. 4,880,635 or U.S. Pat. No. 5,147,652 can be used. See also Yarosh, D., et al., J. Invest. Dermatol., 103(4):461-468 (1994) or Caplen, N. J., et al., Nature Med., 1(1):39-46 (1995).

The liposomes can specifically target the appropriate cells (e.g., epidermal melanocytes). In a preferred embodiment of the invention, the liposomal composition is applied directly to the skin or hair of a mammal, in the area where decreased pigmentation is desired.

The present invention also encompasses methods of identifying an agent that decreases pigmentation in melanocytes. These methods identify a BMP-4 fragment or mimic based on the ability of the BMP-4 fragment or mimic to decrease melanin synthesis by activating the same signaling pathway as activated by BMP-4. A BMP-4 active fragment or mimic, or an active fusion protein of BMP-4, is defined as being able to activate BMP type 1 and type 2 receptors, which can be manifested, for example, by the capacity to induce alkaline phosphatase production in chondrogenic cells, for example, ATDL-5 chondrogenic cells, in addition to having an effect in melanocytes on melanin content, melanin synthesis, NF-κB location, tyrosinase mRNA, tyrosinase protein, PKC-β mRNA, PKC-β protein, microphthalmia transcription factor or any combination of these effects on melanocytes or melanoma cells, or other cells appropriate for the observation of such effects, as described herein.

For example, vertebrate melanocytes are grown in culture under conditions suitable for maintaining growth and viability of the melanocytes. In one embodiment of the invention, the melanocytes are isolated from human newborn foreskin samples. The BMP-4 fragment or mimic to be tested (i.e., the agent to be tested for its effect on cells) is then introduced into the culture and thus, is allowed to interact with the cultured cells. The culture containing the agent to be tested is maintained under conditions suitable for the agent to be tested as a BMP-4 fragment or mimic to affect pigmentation, e.g., to affect melanin synthesis. Control cultures of melanocytes are also maintained under similar conditions but without the BMP-4 fragment or mimic to be tested. After a suitable period of time, the melanocytes are removed from the culture, e.g., by trypsinization or scraping, and the melanin content is quantified.

In a particular embodiment of the invention, the rate of melanin synthesis is measured in melanocyte cultures. This is accomplished by determining the melanin concentration in a test sample of melanocytes at various time intervals for a specified amount of time. The measured melanin content versus time may be plotted graphically in order determine the rate of melanin synthesis.

In other embodiments of the invention, similar procedures for assessing the effect of a BMP-4 fragment or mimic on melanocytes can be performed on melanin-producing melanoma cells as a model of melanocytes.

Measuring the amount of melanin typically encompasses quantifying the amount of melanin synthesis that has occurred while the melanocytes were cultured with the agent to be tested. The melanin content of the cells can be measured directly, for example, as described, in Gordon, P. R. and Gilchrest, B. A., J. Invest. Dermatol., 93:700-702 (1989), the teachings of which are incorporated herein by reference. Briefly, human melanocytes are cultured under standard laboratory conditions. 1×10⁶ cells can be routinely used to measure melanin content. Cells are spun at 2,500 rpm in a centrifuge for 15 minutes and the resulting pellet is dissolved in 0.5 ml of 1 N NaOH. Melanin concentration is calculated by measuring OD₄₇₅ by spectrophotometer and comparing with a standard curve of OD₄₇₅ measurements made using known concentrations of melanin. The melanin content of the melanocytes cultured in the presence of the test agent is compared to the melanin content of control cells cultured in the absence of the test agent. A lower amount of melanin in the melanocytes cultured with the agent is an indication that the agent to be tested inhibits melanin synthesis.

As the level of tyrosinase activity in melanin-producing cells is correlated with the level of melanin production, tyrosinase amounts or activity can be measured to determine melanin production in cells. Thus, an agent that mimics BMP-4 in its suppression of melanin production can be found by testing for a reduction in tyrosinase amount or activity in cultures of cells that respond to BMP-4 by activating the BMP-4 signalling pathways and reducing production of tyrosinase-specific mRNA and tyrosinase protein. The method to test the agent involves contacting the cells in culture with the agent, wherein the cells produce tyrosinase and produce BMP receptor type 1A, BMP receptor type 1B and BMP receptor type 2, measuring the amount or activity of tyrosinase in the cells, and measuring the amount or activity of tyrosinase in control cells. If the amount or activity of tyrosinase per cell measured in the cells contacted with the agent is less than the amount or activity of tyrosinase per cell measured in control cells, then the agent decreases melanin production in the cells. If the amount or activity of tyrosinase per cell measured in cells contacted with the agent is greater than the amount or activity of tyrosinase per cell measured in control cells, then the agent increases melanin production in the cells.

The cells used to test the effect of an agent, using tyrosinase amount or activity as a measure of melanin production, can be melanocytes or the cells of melanoma cell lines the produce conveniently measurable levels of melanin, for example. The concept of control cells is well understood in the art. Control cells in this case can be, for instance, cells of the same genetic lineage which are not exposed to the agent, but other types of negative controls are possible. A positive control in which BMP-4 is tested as the agent can also be useful for comparison.

Tyrosinase amounts can be assessed using immunological assays employing antibodies to the protein, for example, in a western blot performed on a gel used to separate the proteins of the cells. Tyrosinase can also be quantitated by ELISA assay, for example, by lysis of the cells in culture (e.g., in microtiter plates) and measurement of the tyrosinase in the lysate by the use of sandwich ELISA with antibodies against tyrosinase.

Assays analogous to those measuring tyrosinase amounts can be applied to microphthalmia-associated transcription factor to assess the effect of an agent on melanin production in cells. An agent that mimics BMP-4 in its suppression of melanin production can be found by testing for a reduction in the amount of microphthalmia-associated transcription factor in cultures of cells that respond to BMP-4 by activating the BMP-4 signalling pathways. The method to test the agent involves contacting the cells in culture with the agent, measuring the amount of microphthalmia-associated transcription factor in the cells, and measuring the amount of microphthalmia-associated transcription factor in control cells. If the amount of microphthalmia-associated transcription factor per cell measured in the cells contacted with the agent is less than the amount of microphthalmia-associated transcription factor per cell measured in control cells, then the agent decreases melanin production in the cells. If the amount of microphthalmia-associated transcription factor per cell measured in cells contacted with the agent is greater than the amount of microphthalmia-associated transcription factor per cell measured in control cells, then the agent increases melanin production in the cells.

The cells used to test the effect of an agent, using amount of microphthalmia-associated transcription factor as a measure of melanin production, can be melanocytes or the cells of melanoma cell lines, for example, or other cells that respond to BMP-4 by activating the BMP-4 signalling pathways. Control cells in this case can be, for instance, cells of the same genetic lineage which are not exposed to the agent, but other types of negative controls are possible, as would be known by one of skill in the art. A positive control in which BMP-4 is tested as the agent can also be useful for comparison.

Assays similar to those for measuring tyrosinase amounts can be used to measure amounts of microphthalmia-associated transcription factor. MITF can be assayed using immunological assays employing antibodies to the protein, for example, in a western blot performed on a gel used to separate by electrophoresis the proteins of the cells. MITF can also be quantitated by ELISA assay, for example, by lysis of the cells in culture (e.g. in microtiter plates) and measurement of the MITF in the lysate by the use of sandwich ELISA with antibodies against MITF.

One method of the invention involves screening for agents, utilizing cells that express the same BMP-4 receptors as those expressed by melanocytes, and then determining if the agent activates the same signaling that is activated by BMP-4. For example, activation of Smad intracellular protein by BMP-4 causes translocation of the Smad protein into the nucleus upon ligand binding to the BMP-4 receptor (Attisano et al., Curr. Opin. Cell Biol. 12:235-245 (2000)). An agent that facilitates Smad migration into the nucleus of the melanocyte binds to the BMP-4 receptors and is therefore a BMP-4 mimic. Additionally, cells may be transfected with vectors encoding BMP receptors (Nohe et al., J. Biol. Chem. 277(7):5330-8 (2002)). The effects of agents on the BMP-4 signaling pathway may then be studied to determine if the agent is a BMP-4 mimic. Melanoma cells may also be utilized in screening assays because they express the same BMP receptors as found on melanocytes. See Examples 11 and 12.

Specifically encompassed by the present invention are peptides, peptide fragments, proteins, organic or inorganic molecules that mimic the sites of interaction between BMP-4 and its transmembrane receptors, BMP receptors 1A, 1B and 2. These molecules mimic BMP-4 in binding to the BMP-4 receptors and therefore inhibit the PKC-β activation of tyrosinase, thereby decreasing melanin synthesis in melanocytes.

The BMP-4 mimics used in the methods described herein can be e.g., proteins, polypeptides or peptides (comprising natural and/or non-natural amino acids). BMP-4 mimics can also be peptide analogs comprising peptide and non-peptide portions. Such peptides can be constructed with D-isomers rather than the native L-isomers of the amino acids, to increase their resistance to proteolytic degradation within living cells. BMP-4 mimics can also be other organic or inorganic molecules having the biological effect of BMP-4 in decreasing melanin synthesis in melanocytes. BMP-4 mimics can be produced synthetically, for example, after being designed through molecular modeling of BMP-4 analogs with BMP-4 receptors. BMP-4 mimics can also be identified through screening tests for the desired biological and/or binding properties (e.g., ability to decrease melanin synthesis in melanocytes, as well as the ability to induce alkaline phosphatase production in chondrogenic cells). Molecules from natural or synthetic sources can be used in the screening methods. All BMP-4 mimics used in these methods have specific characteristics pertaining to biological activity. These characteristics include the ability of these mimics to bind to the surface of melanocytes or melanoma cells and to cause a decrease in melanin production, tyrosinase expression or PKC-β expression in these cells.

In methods described herein, candidate BMP-4 mimics to be tested for biological activity are referred to as “agents.” In these methods, especially for preliminary screening, more than one candidate BMP-4 mimic can be tested at a time. Thus, “agent” includes the plural meaning as well as the singular.

In one embodiment of the invention, the BMP-4 mimics inhibit PKC-β-mediated phosphorylation of tyrosinase and thus prevent activation of tyrosinase in epidermal melanocytes. The BMP-4 mimics bind to cell surface receptors necessary to prevent the signaling that is responsible for melanin production. This results in a decrease in the production of melanin pigment in melanocytes.

In another embodiment of the invention, the cellular location of NF-κB can be determined after addition of the candidate BMP-4 mimic. The binding of BMP-4 to its receptors induces a set of secondary messengers (such as NF-κB) that act as transcription activators for certain genes (Mohan et al., 1998). NF-κB is normally localized in the cytoplasm of a cell. After stimulation with BMP-4 and activation of the BMP-4 receptors, NF-κB localizes to the nucleus. (Mohan et al., 1998). The cellular location of NF-κB is therefore an indicator of BMP-4 activity. Briefly, melanocytes can be cultured in the presence of the candidate BMP-4 mimic under conditions suitable to maintain the growth and viability of the melanocytes. The cells are subsequently stained with anti-NF-κB antibodies containing a label, and the antibodies are detected using immunohistochemical techniques well known to one of skill in the art. The stained cells are then examined microscopically to determine the location of the bound antibody in the cell. The presence of the antibody in the nucleus indicates that NF-κB has localized to this area as a result of BMP-4 receptor activation. An agent that causes NF-κB localization to the nucleus is a BMP-4 mimic, as it exhibits binding to the melanocyte surface characteristic of BMP-4 (Mohan et al., 1998).

The present invention also encompasses identifying BMP-4 mimics by utilizing cells comprising one or more genetic constructs (e.g., in a vector), the construct comprising a BMP-4 responsive promoter located (for DNA transcription) upstream of, and controlling transcription of, a reporter gene. As used herein, a “BMP-4 responsive promoter” is a promoter which is activated or repressed following the binding of an agent (e.g., BMP-4, active fragment thereof, or BMP-4 mimic) to the BMP transmembrane receptors. Binding of BMP-4 activates the BMP-4 receptors, which induce a set of secondary messengers. BMP-4, like other members of the BMP family, binds to type 2 and type 1 serine/threonine kinase receptors and transduces intracellular signals through Smad proteins. Certain Smads form complexes with Co-Smad proteins and the complex translocates into the nucleus, where it regulates transcription of various target genes (Massague et al., 2000; Attisano, et al., 2000). Another example of a secondary messenger induced by activation of BMP-4 receptors is NK-κB. As used herein, a reporter gene is a gene that encodes an easily assayed product (e.g., chloramphenicol acetyl transferase (CAT), luciferase, green fluorescent protein, β-galactosidase) wherein the coding region of the reporter gene is coupled to the upstream transcription control region of another gene. Expression of the reporter gene can then be used to determine which factors activate or repress response elements by binding the promoter region controlling transcription in the reporter gene construct.

Cells of melanocytic lineage (melanocytes and melanoma cells) can be transfected with a vector containing a gene comprising a BMP-4 responsive promoter located upstream of and operably linked to a reporter gene. The transformed cells are then contacted with the candidate agent being tested as a BMP-4 mimic. The amount of reporter gene expression in the transfected culture of cells is then measured and compared to the amount of the reporter gene expression in one or more control cultures of cells, for instance transfected cultures of cells which have not been contacted with the agent. Binding of the BMP-4 mimic activates BMP receptors, inducing activity of intracellular proteins which regulate the BMP-4 responsive promoter. The BMP-4 responsive promoter can be, in certain embodiments, the tryosinase promoter or the MITF promoter. The promoter drives the expression of the downstream reporter gene, and the gene product can be quantified. An increased/decreased amount of reporter gene expression observed at one or more times after contact with the agent indicates that the agent is increasing/decreasing transcription from the BMP-4 responsive promoter and the agent is therefore a BMP-4 mimic. The gene product most conveniently assayed is the protein, but the gene product to be assayed can also be the mRNA.

Cells other than melanin-producing cells can also be used to identify an agent that reduces melanin production in melanin-producing cells. Such cells comprise in their cell membranes functional BMP receptor type 1A, BMP receptor type 1B and BMP receptor type 2, and the cells further comprise one or more genetic constructs comprising a BMP-4 responsive promoter operably linked to a reporter gene. Receptors for BMP-4 are found in osteoblasts and osteoblastic cell lines (Ten Kijke, P., et al., J. Biol. Chem. 269:16985-16988, 1994), in NIH 3T3 cells (Koenig, B. B. et al., Mol. Cell. Biol. 14:5961-5974, 1994) and in skin fibroblasts (Nohno, T. et al., J. Biol. Chem. 270:22522-22526, 1995). Further, it is possible to introduce BMP-4 receptors into cells that do not endogenously produce them. See, for example, Nohe A. et al., J. Cell Sci. 116:3277-3284, 2003, describing the transfection of COS7 and A431 cells with plasmids encoding BMP receptors. Genetic constructs comprising a BMP-4 responsive promoter operably linked to a reporter gene can be introduced into any cells bearing receptors for BMP-4, and the resulting cells can be used to identify agents that down-regulate tyrosinase production, and therefore, melanin production, in melanin-producing cells.

BMP receptor type 1A, BMP receptor type 1B and BMP receptor type 2 are functional receptors if they can be demonstrated to be present on the cells and if one or more intracellular events characteristic of BMP-4 activity as known in the art can be shown in response to BMP-4 addition to the cells.

The methods of the invention can be used to identify BMP-4 mimics by their ability to down regulate PKC-β, and thus phosphorylation of tyrosinase, in cells of melanocytic lineage. Binding of the BMP-4 mimic to the surface of the cells inhibits phosphorylation of tyrosinase by PKC-β and thus decreases melanin production. Specifically, cells of melanocytic lineage can be cultured in the presence of the agent being tested as a candidate BMP-4 mimic, under conditions suitable for the growth and viability of the cells. Control cultures of the cells are maintained under similar conditions but without the addition of the agent. The level of PKC-β-specific mRNA in the cells cultured with the agent is determined and compared to the level of RNA from a control culture of cells grown in the absence of the agent. If the agent decreases the level of PKC-β RNA in the cells exposed to the agent as compared to the level of PKC-β RNA in the control, the agent is a BMP-4 mimic. In another embodiment of the invention, samples of cells of melanocytic lineage cultured in the presence of the agent may be taken at various time intervals. Total RNA is then extracted as indicated above, and the level of PKC-β RNA is compared to that in samples of control cells collected at the same time intervals but grown in the absence of the agent.

In one embodiment of the methods of the invention, the presence of PKC-β RNA is determined by northern blot analysis using methods known to one of skill in the art. (See Ch. 4.9 In Current Protocols in Molecular Biology, Ausubel, F. et al., eds., containing supplements up through Supp. 57, John Wiley & Sons, 2002). Briefly, total cellular RNA is isolated from the cells being tested. The RNA is separated by gel electrophoresis and transferred to a membrane. A labeled PKC-β DNA probe is produced using methods known in the art. The label can be, for instance, a radioactive isotope. The membrane is contacted with a solution containing the probe under conditions allowing for hybridization of the probe to the portions of the membrane to which PKC-β RNA is bound. The membranes are washed to eliminate unbound probe and non-specifically bound probe, and the bound probe is then visualized using known techniques, according to the labeling method used for the probe. Binding of saturating amounts of the PKC-β DNA probe to portions of the membrane indicates the presence of PKC-β RNA in the cultured cells being tested. Saturating amount of bound probe may be quantified using conventional techniques, indicating the level of PKC-β in the sample. A similar analysis is performed on control cells cultured in conditions identical to those for the cells contacted with the agent, but grown in the absence of the agent. A decrease in the amount of PKC-β RNA recovered from cells treated with the candidate agent in comparison with the control cells indicates that the candidate agent decreases PKC-β production and thus decreases melanin synthesis. The agent is then identified as a BMP-4 mimic.

Additionally, the methods of the invention are directed to identifying BMP-4 mimics by their ability to down regulate PKC-β in cells of melanocytic lineage, by determining the amount of PKC-β protein isolated from the cells and detected by western blot analysis. Specifically, cells of melanocytic lineage are cultured in the presence of the agent. Total cellular protein is isolated by methods known to those of skill in the art (Zhai, S. et al., Exp. Cell Res. 224:335-343 (1996)). The proteins are separated by gel electrophoresis and transferred to a membrane for western blot (Ch. 10.8 In Current Protocols in Molecular Biology, Ausubel, F. et al., eds., containing supplements up through Supp. 57, John Wiley & Sons, 2002). An anti-PKC-β antibody is diluted in a solution and contacted with the membrane under conditions allowing for binding of saturating amounts of the anti-PKC-β antibody to the portions of the membrane containing PKC-β protein. The membranes are washed to eliminate unbound and non-specifically-bound antibody and then incubated with a secondary antibody (directed against the first antibody) labeled appropriately to permit visualization. The label can be, for example, a radioisotope or fluorophore, or an enzyme such as horseradish peroxidase. Binding of the anti-PKC-β antibody to portions of the membrane indicates the presence of PKC-β protein in the cultured cells. The amount of bound antibody can be quantified using conventional techniques, indicating the level of PKC-β in the cell sample. A similar analysis is performed on control cells cultured in identical conditions but grown in the absence of the agent. A western blot reflecting a lower amount of PKC-β protein in the cells treated with the candidate agent in comparison with the amount of PKC-β protein in the control cells indicates that the candidate agent decreases PKC-β production and thus decreases melanin synthesis. The agent is then identified as a BMP-4 mimic.

### EXAMPLES

### Example 1

### BMP-2/4 Decreases Melanin Production in Melanocytes

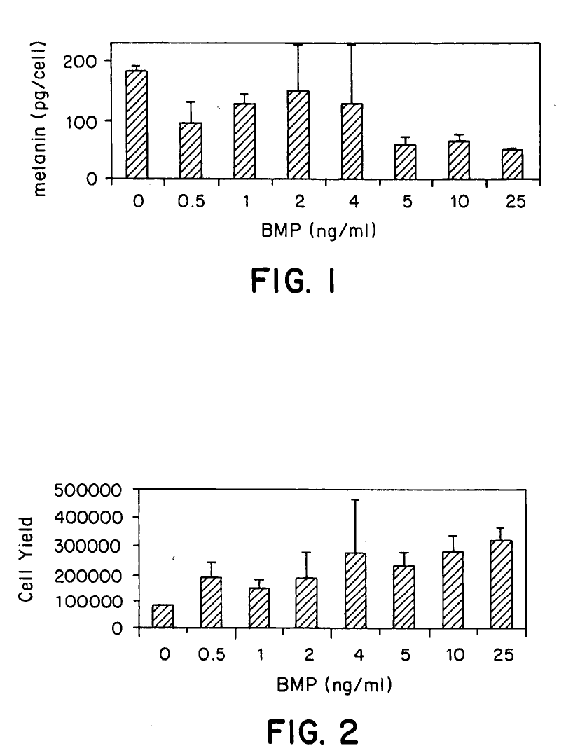

Human newborn melanocytes were isolated from foreskin specimens as described (Gilchrest, B. A. et al., In Vitro Cell & Develop Biol. 21:114-120 (1985)). Briefly, foreskin fragments were rinsed in phosphate buffered saline (PBS) and incubated overnight at 4° C. in 0.25% trypsin. Epidermal sheets were then mechanically separated, placed in 0.02% EDTA, vortexed to yield a single cell suspension and plated at 10⁶ cells/35 mm dish. Cells were maintained in serum free, growth factor supplemented and hormone supplemented medium as described (Yaar, M. et al., J. Clin. Invest. 100:2333-2340 (1997)). Briefly, cells were maintained in medium 199, supplemented with 10 ng/ml epidermal growth factor (EGF), 10 μg/ml insulin, 10⁻⁹ M triiodothyronine, 10 μg/ml transferrin, 1.4×10⁻⁶ M hydrocortisone, 10 ng/ml basic fibroblast growth factor and 10 μg/ml inositol, in the presence of increasing concentrations of BMP-2/4 or diluent (diluent shown as “0” in FIG. 1) as control. BMP-4 activity of the BMP-2/4 fusion protein was measured by its ability to induce alkaline phosphatase production in ATDL-5 chondrogenic cells. The ED₅₀ for this effect is routinely at 10-30 ng/ml. Cells were harvested 72 hours after addition of BMP-2/4. Paired dishes were trypsinized, washed twice with PBS, and solubilized in 0.8 ml 1 N NaOH with vigorous vortexing for 10-15 minutes. Melanin concentration was determined by measurement of OD₄₇₅ and comparison with a standard curve obtained using a synthetic melanin (Sigma). Results in FIG. 1 are expressed as melanin content per cell. BMP-2/4 (25 ng/ml) decreased melanin content by approximately 75%. Cell yields of duplicate cultures increased as a result of BMP-2/4 supplementation (FIG. 2).

### Example 2

### BMP-2/4 Does Not Induce Melanocyte Apoptosis

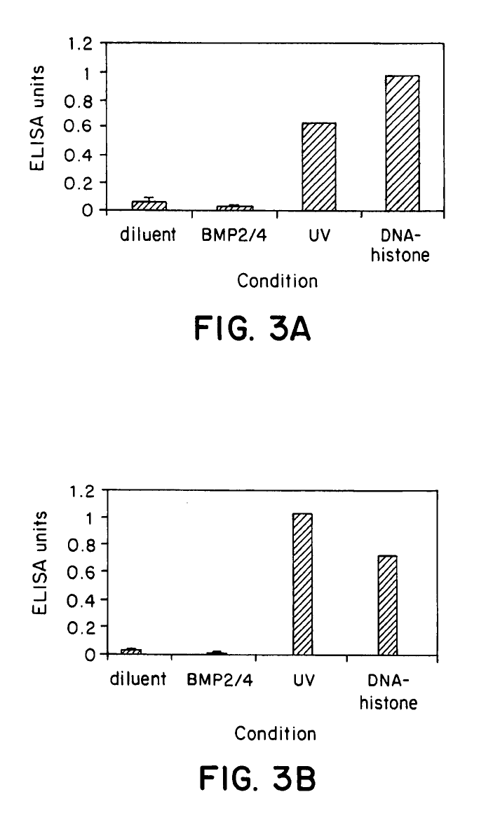

To rule out the possibility that BMP-2/4 is toxic to melanocytes, cells were maintained as in Example 1 in the presence of 10 ng/ml BMP-2/4 for 48 to 72 hours. Melanocyte apoptosis was determined using the Cell Death Detection Elisa^{PLUS} kit (Roche, Indianapolis, Ind.), which measures cytoplasmic histone-associated mono- and oligonucleosomes generated as a result of apoptosis. Melanocytes treated with BMP-2/4 did not display apoptosis above the low background level, while cells UV irradiated with 50 mJ/cm of solar simulated irradiation metered at 285±5 nm showed a greater than 100-fold increase in reaction product 72 hours after irradiation, as expected. See FIGS. 3A and 3B. The positive control (DNA-histone complex) included in the kit showed the expected high signal and was included to establish that the different compounds provided in the kit are functional.

### Example 3

### BMP-2/4 Downregulates Tyrosinase mRNA

Melanocytes were maintained as in Example 2 in the presence of 10 ng/ml BMP-2/4 or diluent as control. Total cellular RNA was harvested at 24, 48 or 72 hours after addition of BMP-2/4 or diluent and processed for northern blot analysis. The blots were hybridized to tyrosinase cDNA. The cDNA probe for human tyrosinase (Pmel34) was a generous gift from B. Kwon (Guthrie Research Institute, Sayre, Pa.). In two independent experiments, BMP-2/4 substantially decreased tyrosinase mRNA as early as 15 hours after addition of BMP-2/4.

### Example 4

### BMP-2/4 Downregulates Tyrosinase Protein

Because the maximal effect of BMP-2/4 on melanin was observed at a concentration of 25 ng/ml, melanocytes were maintained as in Example 2 in the presence of 25 ng/ml BMP-2/4 or diluent as control. Total cellular proteins were harvested at 24, 48 or 72 hours after supplementation with BMP-2/4 or diluent and processed for western blot analysis. The blot was reacted with anti-tyrosinase antibodies (Novocastra Laboratories Ltd, New Castle upon Tyne, UK) at a 1:500 dilution. BMP-2/4 substantially decreased tyrosinase protein within 24 hours after supplementation with BMP-2/4. The effects persisted through 48 hours and were still detectable 72 hours after the single stimulation. As expected, the tyrosinase baseline level increased with culture maturity in both treated and control cultures.

### Example 5

### BMP-2/4 Downregulates PKC-β mRNA

Melanocytes were maintained as in Example 1 in the presence of 25 ng/ml BMP-2/4 or diluent as control. Total cellular RNA and proteins were harvested 24 hours after addition of BMP-2/4 and processed for northern and western blot analyses. The northern blot was hybridized to PKC-β cDNA. The cDNA probe specific for PKC-β was purchased from American Type Culture Collection (Manassas, Va.). BMP-2/4 substantially decreased the two known PKC-β transcripts (4.2 and 10 kb) as reported (Yamanishi, D. T. et al., arcinogenesis 12:105-109 (1991); Park, H. Y. et al., J. Biol. Chem . 268:11742-11749 (1993)). The western blot reacted with anti-PKC-β antibodies showed that BMP-2/4 substantially decreased the level of PKC-β protein. Anti PKC-β antibodies were obtained from Transduction Laboratories (Lexington, Ky.) and were used at 25 ng/ml.

### Example 6

### Tyrosinase Promoter Includes the Consensus Sequence for BMP-4

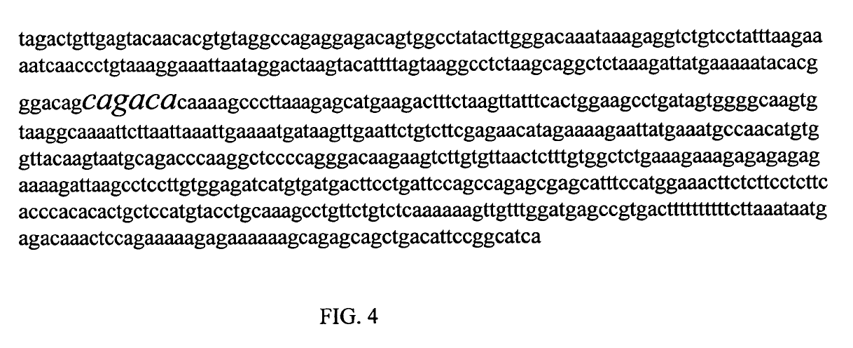

BMP-4, like other members of the BMP family, binds to type 2 and type 1 serine/threonine kinase receptors and transduces intracellular signals through Smad proteins. Certain Smads form complexes with Co-Smad proteins and the complex translocates into the nucleus, where it regulates transcription of various target genes (reviewed in Massague et al., 2000; Attisano et al., 2000)). Transfection experiments using the BMP-responsive type X collagen promoter upstream of and operably linked to the luciferase gene resulted in luciferase expression within 24 and 48 hours of BMP stimulation, suggesting that gene transcription as a result of BMP stimulation occurs within this time frame (Leboy, P. et al., J. Bone Joint Surg Am. 83-A, Suppl 1:S15-22 (2001)). One of the consensus sequences recognized by the Smad/Co-Smad transcription factor is CAGACA of the human tyrosinase promoter as published by Ponnazhagan et al., J. Invest. Dermatol. 102:744-748 (1994). The tyrosinase promoter includes this consensus sequence. Shown in FIG. 4 is part of the tyrosinase promoter with the consensus sequence for Smad/Co-Smad in bold, obtained from National Center for Biotechnology Information (NCBI) (www.ncbi.nlm.nih.gov/entrez/query, accession # U03039) (Ponnazhagan et al., J. Invest. Dermatol 102:744-748 (1994)).

### Example 7

### Melanocytes Express BMP Receptor Type 1A

Total melanocyte RNA was harvested from normal human melanocytes maintained as in Example 1. cDNA was generated by reverse transcription and putative BMP receptor type 1A specific DNA was amplified by PCR using primers complementary to the human BMP receptor type 1A mRNA (Mohan, et al., 1998).

| | |
|---|---|
| Upstream Primer: | |
| ^{5′}GGACATTGCTTTGCCATCATA^{3′} | (SEQ ID NO:2) |
| Downstream Primer: | |
| ^{5′}CAGACCCACTACCAGAACTTT^{3′} | (SEQ ID NO:3) |

The PCR products were separated by electrophoresis on a 1% agarose gel, and a strong band was identified. The band was sequenced and the DNA sequence was compared to the working draft sequence of the human genome (BLAST at http://www.ncbi.nlm.nih.gov/genome/seq/). The sequenced band was identified as having the coding sequence for human BMP receptor 1A.

### Example 8

### Melanocytes Express BMP Receptor Type 1B

Total melanocyte RNA was harvested from normal human melanocytes maintained as in Example 1. cDNA was generated by reverse transcription and putative BMP receptor type 1B specific DNA was amplified by PCR using primers complementary to the human BMP receptor type 1B mRNA (Mohan, R. R. et al., 1998).

| | |
|---|---|
| Upstream Primer: | |
| ^{5′}GTTGTAAATGCCACCACCATT^{3′} | (SEQ ID NO:4) |
| Downstream Primer: | |
| ^{5′}GTCTGGTTTCTTGTCTTTTAT^{3′} | (SEQ ID NO:5) |

The PCR products were separated by electrophoresis on a 1% agarose gel and a single band was identified. The band was sequenced and the DNA sequence was compared to the working draft sequence of the human genome (BLAST at http://www.ncbi.nlm.nih.gov/genome/seq/). The sequenced band was identified as having the coding sequence for human BMP receptor 1B.

### Example 9

### Melanocytes Express BMP Receptor Type 2

Total melanocyte RNA was harvested from normal human melanocytes maintained as in Example 1. cDNA was generated by reverse transcription and putative BMP receptor type 2 specific DNA was amplified by PCR using primers complementary to the human BMP receptor type 2 mRNA (Mohan, R. R. et al., 1998).

| | |
|---|---|
| Upstream Primer: | |
| ^{5′}TGGCTGAACTTATGATGATTT^{3′} | (SEQ ID NO:6) |
| Downstream Primer: | |
| ^{5′}TGTTGGTGGAGAGGCTGGTGA^{3′} | (SEQ ID NO:7) |

The PCR products were separated by electrophoresis on a 1% agarose gel and a strong band was identified. The band was sequenced and the sequence was compared to the working draft sequence of the human genome (BLAST at http://www.ncbi.nlm.nih.gov/genome/seq/). The sequenced band was identified as having the coding sequence for human BMP receptor 2.

### Example 10

### NF-κB in Melanocytes is Activated Upon BMP-4 Stimulation

Melanocytes were obtained and maintained as in Example 1, and were stimulated with 25 ng/ml BMP-4 or with diluent as control for 10 minutes. Cultures were processed for NF-κB staining using immuno-histo-chemical techniques. Anti-NF-κB antibodies were obtained form Santa Cruz Biotech (Santa Cruz, Calif., NF-κB, p 65, F-6) and were used at a 1:200 dilution. In cells to which diluent was added, NF-κB was localized to the cytoplasm and was particularly found in perinuclear distribution. Within 10 minutes of BMP-4 stimulation, NF-κB localized to the nucleus, proving receptor activation.

### Example 11

### Melanoma Cells Express BMP Receptor Type 1A and BMP Receptor Type 1B

Three melanoma cell lines (AN, EP and LH) were maintained in DME Media supplemented with 10% fetal bovine serum. Total cellular RNA was harvested. cDNA was generated by reverse transcription and was amplified by PCR with primers complementary to the human BMP receptor type 1A mRNA, and in separate tubes cDNA was used as a template to amplify DNA encoding BMP receptor type 1B (Mohan, R. R. et al., Invest. Ophthalmol. Vis. Sci. 39:2626-2636 (1998)).

| | |
|---|---|
| For BMP receptor type 1A: | |
| Upstream primer: | |
| ^{5′}GGACATTGCTTTGCCATCATA^{3′} | (SEQ ID NO:2) |
| Downstream primer: | |
| ^{5′}CAGACCCACTACCAGAACTTT^{3′} | (SEQ ID NO:3) |
| For BMP receptor type 1B: | |
| Upstream Primer: | |
| ^{5′}GTTGTAAATGCCACCACCATT^{3′} | (SEQ ID NO:4) |
| Downstream Primer: | |
| ^{5′}GTCTGGTTTCTTGTCTTTTAT^{3′} | (SEQ ID NO:5) |

PCR products were separated over a 1% agarose gel and strong bands at the expected molecular weights were identified in all melanoma cell lines, indicating that melanoma cells express BMP receptor type 1A and 1B.

### Example 12

### Melanoma Cells Express BMP Receptor Type 2

Three melanoma cell lines (AN, EP and LH) were maintained as in Example 11. Total cellular RNA was harvested. cDNA was generated by reverse transcription and was amplified by PCR using primers complementary to the human BMP receptor type 2 mRNA (Mohan, R. R. et al.).

| | |
|---|---|
| Upstream primer: | |
| ^{5′}TGGCTGAACTTATGATGATTT^{3′} | (SEQ ID NO:6) |
| Downstream Primer: | |
| ^{5′}TGTTGGTGGAGAGGCTGGTGA^{3′} | (SEQ ID NO:7) |

PCR products were separated over a 1% agarose gel and a strong band at the expected molecular weight was identified in all melanoma cells, indicating that melanoma cells express BMP type 2 receptor.

### Example 13

### BMP-2/4 Represses Tyrosinase Promoter Activity

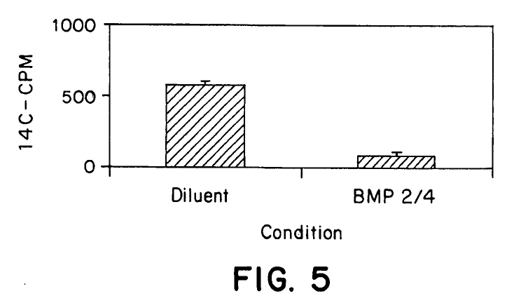

4×10⁴ AN melanoma cells were plated in 60 mm dishes. Twenty-four hours after plating, cells were transfected with a tyrosinase-CAT plasmid construct carrying the full tyrosinase promoter (6.1 kb) (Kluppel, M. et al., Proc. Natl. Acad. Sci. USA 88:3777-3781 (1991)) using the FuGENE™ 6 transfection reagent (Roche Applied Science) and following the recommendation of the manufacturer. At the time of transfection, cells were 50-80% confluent. Twenty-four hours after transfection, cells were stimulated with BMP-2/4 (25 ng/ml). CAT activity was determined 72 hours after transfection. One representative experiment is shown in FIG. 5. In a total of 2 experiments, BMP-2/4 decreased CAT activity by 48 +16% (mean+SEM), showing that BMP-2/4 transcriptionally represses tyrosinase promoter activity.

### Example 14

Since UV irradiation is the best-recognized environmental stimulator of melanogenesis, the effect of UV irradiation on BMP-4 expression in both keratinocytes and melanocytes was examined, and the level of BMP-4 receptors in melanocytes was determined.

### A) UV Irradiation Downregulates BMP Receptors on Melanocytes

Subconfluent normal human melanocytes were irradiated in PBS using a solar simulator (Spectral Energy Corporation, Westwood, N.J.) housing an appropriately filtered 1 kW xenon arc lamp (XMN 1000-21; Optical Radiation Corp., Azusa, Calif.) adjusted to 2×10⁴ W cm⁻². This system delivers a spectral output virtually identical to that of terrestrial sunlight (Werninghaus, K. et al., Photodermatol Photoimmunol Photomed 8:236-242 (1991)). Dosage was metered at 285±5 nm with a research radiometer (model IL1700A; International Light Inc., Newburyport, Mass.) fitted with a UVB probe (detector SSE 240, diffuser W, filter UVB). Dose was calculated to contain 20 mJ/cm². Sham irradiated cells were handled identically but were placed under an aluminum foil cover during the irradiation. After irradiation, cells were provided fresh medium and were harvested 24 hours later. The dose of UV irradiation used is physiologically relevant and constitutes an exposure expected to result in moderately severe sunburn.

UV irradiation substantially downregulated the mRNAs of the three BMP receptors in melanocytes as determined by RT-PCR, consistent with the hypothesis that BMP-4 decreases melanogenesis and hence after UV irradiation, when pigmentation is expected to be induced and tyrosinase levels increased, the levels of the receptors for BMP-4, a physiologic factor that inhibits tyrosinase transcription, are decreased.

### B) UV Irradiation Downregulates BMP-4 Expression in Melanocytes and Keratinocytes

Subconfluent normal human melanocytes and keratinocytes were irradiated with 20 mJ/cm² measured at 285±5 nm in PBS as above. Sham irradiated cells were handled identically but were placed under an aluminum foil cover during the irradiation. After irradiation, cells were provided fresh medium and total cellular RNA was harvested 24 hours after irradiation (melanocytes) and up to 72 hours after irradiation (keratinocytes). UV irradiation substantially downregulated BMP-4 mRNAs in both keratinocytes and melanocytes as confirmed by RT-PCR, consistent with the hypothesis that after UV irradiation, when pigmentation is expected to be induced and tyrosinase levels increased, the levels of autocrine and paracrine BMP-4, a physiologic factor that inhibits tyrosinase transcription, are decreased.

### Example 15

### Generation of Transgenic Mice

C3H/HeJ mice were purchased from the Jackson Laboratory (Bar-Harbor, Me.) and were used as a background strain for generating K5-Noggin overexpressing mice. A K5-Noggin transgene was constructed using the plasmid vector pGEM 3Z with 1690 bp human K5 promoter, 984 bp Flag-noggin sequence, and polyadenylation sequence. The Flag sequence was inserted at the 5′ end of the full length mouse noggin cDNA. This cDNA was inserted into the expression cassette between the 1.69 kb human K5 promoter containing beta-globin intron and a transcription termination/polyadenylation [poly(A)] fragment of the human K14 gene. The 3.2 kb insert fragment was cleaved from the plasmids with EcoRI and HindIII and then purified for microinjections, using a CsCl gradient. The K5-Noggin construct was injected into the fertilized eggs of F1 C3H/HeJ mice by the Transgenic Animal Facility at Boston University. Two transgenic founders were identified, and the F₂ populations of mice derived from two transgenic lines were generated. K5-Noggin mice are viable and fertile, and develop postnatally the switch in hair pigmentation from yellow-brown to brown-black color.

### Example 16

### BMP-2/4 Role in Modulating Hair Color

Pheomelanin (red/yellow) and eumelanin (brown/black) pigments can be found in the fur, wool or hair of mammals. In C3H/HeJ mice, the yellow-brown color of hairs is determined by tightly coordinated regulation of pheo- and eu-melanogenesis in hair follicle melanocytes during the hair cycle. In early anagen hair follicles, melanocytes produce eumelanin leading to appearance of the brown pigment at the tip of the hair. Then, on days 5-6 after hair cycle initiation, melanocytes begin to produce pheomelanin due to the inhibition of signaling through the melanocortin-1 receptor (MC-1R) by the agouti signaling protein (ASP), which is produced by dermal papilla fibroblasts and prevents the interaction of a- melanocyte stimulating hormone with MC-1R (Millar et al., Development 121: 3223-3232, (1995)). This leads to the appearance of yellow band closely located to the tip of the hair. During the hair cycle progression, the expression of ASP is decreased leading to the increase of signaling through MC-1R. This results in the switch of pigment production from pheomelanin to eumelanin by follicular melanocytes and in the appearance of brown pigment at the central and proximal portions of the hair.

Hair pigmentation phenotype seen in K5-Noggin mice suggests that the yellow pigment at the distal part of the hair is replaced by brown-black pigment resulting in brown-black color of the fur. This suggests that BMP signaling is involved in the control of expression of the proteins regulating pheo- and eu-melanogenesis during the hair cycle. Using western blot analysis, we found that the levels of agouti signaling protein in full-thickness skin lysates obtained on day 5 of a depilation-induced hair cycle is markedly reduced in K5-Noggin mice, compared to age-matched wild-type mice. This corresponds well with the presence of Smad1- and Smad5-specific sequences on the promoter of agouti gene and suggests that agouti gene may represent a new target for BMP regulation in the hair follicle. Taken together, these data suggest that BMP-2/4 may not only control the intensity of hair color (lighter/darker), but also modulate or change hair coloration via controlling the expression of agouti signaling protein.

### Example 17

### BMP-4 Decreases the Level of Microphthalmia-Associated Transcription Factor (MITF) Protein

Paired cultures of human melanocytes were treated with vehicle or BMP-4 (25 ng/ml media) for 24 and 48 hours. Cells were harvested at the indicated times and processed for immunoblot analysis using a monoclonal antibody against human MITF, according to the following procedures.

Total cellular proteins were collected in RIPA buffer consisting of 0.25 M Tris HCl (pH 7.5), 0.375 M NaCl, 2.5% sodium deoxycholate, 1% Triton X-100, 25 mM MgCl₂, 1 mM phenylmethyl sulfonyl fluoride, and 0.1 mg/ml aprotinin. Protein concentrations were determined by the Bradford method and 10 to 100 μg protein/lane were separated over 7.5% SDS-PAGE and blotted onto nitrocellulose (overnight, 40V). Blots were incubated with the primary antibody followed by horseraddish peroxidase-conjugated secondary antibody. Antibody binding was detected by the ECL detection kit (Amersham, Piscataway, N.J.), followed by autoradiography (Kodak X-Omatic AR). Antibody against human tyrosinase was pruchased from Novacostra Laboratories Ltd. and the monolonal antibody against MITF was kindly provided by Dr. David Fisher at the Dana Farber Cancer Institute. BMP-4 was recombinant human BMP-2/4 protein purchased from R & D Systems (Minneapolis, Minn.).

The levels of both phosphorylated and non-phosphorylated MITF were decreased after 24 and 48 hours of BMP-4 treatments, as seen from the immunoblot.

While this invention has been particularly shown and described with references to preferred embodiments thereof, it will be understood by those skilled in the art that various changes in form and details may be made therein without departing from the scope of the invention encompassed by the appended claims.

1-9. (canceled)

10. A method of identifying a fragment of BMP-4 or a mimic of BMP-4 that inhibits synthesis of melanin in cells of melanocytic lineage, said method comprising:
a) culturing cells of melanocytic lineage in the presence of the fragment or mimic;
b) measuring the melanin content in the cells; and
c) comparing the melanin content to the melanin content measured in control cells not cultured in the presence of the fragment or mimic;
wherein a lower melanin content in the cells of a) than the melanin content in the control cells is indicative that said fragment or mimic inhibits synthesis of melanin in cells of melanocytic lineage.

11. A method of identifying a BMP-4 fragment or mimic that inhibits synthesis of melanin in cells of melanocytic lineage, said method comprising:
a) adding the fragment or mimic to cells of melanocytic lineage in culture;
b) measuring the change in melanin content after addition of the fragment or mimic to the cells; and
c) comparing the change in melanin content to any change in melanin content in control cells to which the fragment or mimic is not added;
wherein a decrease in the melanin content in the cells of melanocytic lineage after addition of the fragment or mimic is indicative that said fragment or mimic inhibits synthesis of melanin in cells of melanocytic lineage.

12. The method of claim 11 wherein the cells of melanocytic lineage are melanocytes isolated from newborn humans.

13. A method of identifying an agent that mimics the activity of BMP-4 in cells of melanocytic lineage, said method comprising:
a) incubating cells of melanocytic lineage with the agent in culture; and
b) assaying for NF-κB in the nuclei of the cells;
wherein NF-κB in the nuclei at a concentration higher than the concentration of NF-κB in control cells of melanocytic lineage not incubated with the agent in culture is indicative that said agent mimics the activity of BMP-4 in cells of melanocytic lineage.

14. A method of identifying an agent that mimics the activity of BMP-4 on cells of melanocytic lineage, said method comprising:
a) transfecting cells of melanocytic lineage with a vector comprising a BMP-4 responsive promoter operably linked to a reporter gene, thereby producing transfected cells of melanocytic lineage;
b) contacting said transfected cells of melanocytic lineage with the agent; and
c) measuring the amount of gene product produced as a result of reporter gene expression in the transfected cells contacted with the agent and in transfected control cells not contacted with the agent;
wherein a difference in the amount of gene product produced as a result of reporter gene expression in said transfected cells contacted with the agent compared to the amount of gene product in transfected control cells not contacted with said agent is indicative that said agent mimics the activity of BMP-4 on cells of melanocytic lineage.

15. (canceled)

16. A method for identifying a BMP-4 fragment or mimic which decreases the level of melanin in cells of melanocytic lineage, said method comprising:
a) incubating the BMP-4 fragment or mimic with cells of melanocytic lineage in culture;
b) determining the level of tyrosinase mRNA in the cells; and
c) comparing the level of tyrosinase mRNA determined in b) with a level of tyrosinase mRNA determined in control cells not incubated with the BMP-4 fragment or mimic;
whereby, if the level of tyrosinase mRNA determined in b) is lower than the level of tyrosinase mRNA determined in the control cells, then the BMP-4 fragment or mimic decreases the level of melanin in the cells of melanocytic lineage.

17. A method for identifying a BMP-4 fragment or mimic which decreases the level of melanin in cells of melanocytic lineage, said method comprising:
a) incubating the BMP-4 fragment or mimic with cells of melanocytic lineage in culture;
b) determining the level of PKC-β RNA in the cells and;
c) comparing the level of PKC-β RNA determined in b) with a level of PKC-β RNA determined in control cells not incubated with the BMP-4 fragment or mimic;
whereby, if the level of PKC-β RNA determined in b) is lower than the level of PKC-β RNA determined in the control cells, then the BMP-4 fragment or mimic decreases the level of melanin in the cells of melanocytic lineage.

18. A method for identifying a BMP-4 fragment or mimic which decreases the level of melanin in cells of melanocytic lineage, said method comprising:
a) incubating the BMP-4 fragment or mimic with cells of melanocytic lineage in culture;
b) determining the level of PKC-β protein in the cells; and
c) comparing the level of PKC-β protein determined in b) with a level of PKC-β protein determined in control cells not incubated with the BMP-4 fragment or mimic;
whereby, if the level of PKC-β protein determined in b) is lower than the level of PKC-β protein determined in the control cells, then the BMP-4 fragment or mimic decreases the level of melanin in the cells of melanocytic lineage.

19. A method for identifying a BMP-4 fragment or mimic which decreases the level of melanin in cells of melanocytic lineage, said method comprising:
a) incubating the BMP-4 fragment or mimic with cells of melanocytic lineage in culture;
b) staining the cells of a) with anti-NF-κB antibodies; and
c) comparing the distribution of antibody staining of the cells of step b) with the distribution of antibody staining of control cells not incubated with the BMP-4 fragment or mimic;
wherein, if the distribution of antibody staining of the cells of step b) is predominantly nuclear, compared to predominantly cytoplasmic and perinuclear in the control cells, then the BMP-4 fragment or mimic decreases the level of melanin in cells of melanocytic lineage.

20. A method for identifying a BMP-4 fragment or mimic which decreases the level of melanin in cells of melanocytic lineage, said method comprising:
a) incubating the BMP-4 fragment or mimic with cells of melanocytic lineage in culture;
b) performing northern blot analysis of total cellular RNA isolated from the cells at various time intervals of incubation of the BMP-4 fragment or mimic with the cells, using DNA encoding human tyrosinase as a probe, thereby quantifying tyrosinase RNA; and
c) comparing the results of step b) with results of northern blot analysis performed on total cellular RNA isolated from control cells not incubated with the BMP-4 fragment or mimic;
wherein, if the level of tyrosinase-specific RNA seen in step b) decreases over time of incubation with the BMP-4 fragment or mimic, compared to the level of tyrosinase-specific RNA seen for the control cells, then the BMP-4 fragment or mimic decreases the level of melanin in the cells of melanocytic lineage.

21. A method for identifying a BMP-4 fragment or mimic which decreases the level of melanin in cells of melanocytic lineage, said method comprising:
a) incubating the BMP-4 fragment or mimic with cells of melanocytic lineage in culture;
b) performing northern blot analysis of total cellular RNA isolated from the cells of a) at various time intervals of incubation of the BMP-4 fragment or mimic with the cells of a), using DNA encoding human PKC-β as a probe, thereby quantifying PKC-β-specific RNA; and
c) comparing the results of step b) with results of northern blot analysis performed on total cellular RNA isolated from control cells not incubated with the BMP-4 fragment or mimic;
wherein, if the level of PKC-β-specific RNA seen in step b) decreases over time of incubation with the BMP-4 fragment or mimic, compared to the level of PKC-β-specific RNA seen for the control cells, then the BMP-4 fragment or mimic decreases the level of melanin in the cells of melanocytic lineage.

22. A method for identifying a BMP-4 fragment or mimic which decreases the level of melanin in cells of melanocytic lineage, said method comprising:
a) adding the BMP-4 fragment or mimic to cells of melanocytic lineage in culture;
b) producing an extract of total cellular proteins from samples of the cells taken at several times after addition of the BMP-4 fragment or mimic in step a);
c) separating the proteins of step b) by gel electrophoresis;
d) blotting the proteins of step c) on a membrane for western blot;
e) incubating the membrane of d) with saturating amounts of anti-tyrosinase antibodies, thereby allowing the binding of the antibodies to tyrosinase; and
f) applying a means to detect bound antibodies, thereby quantifying the bound antibodies;
whereby, if the bound antibodies decrease with time after addition of the BMP-4 fragment or mimic to the cells, then the BMP-4 fragment or mimic decreases the level of melanin in the cells of melanocytic lineage.

23-28. (canceled)

29. A method of identifying an agent that reduces melanin production in cells of melanocytic lineage, said method comprising:
a) contacting cells, wherein the cells comprise in their cell membranes functional BMP receptor type 1A, BMP receptor type 1B and BMP receptor type 2, and the cells further comprise one or more genetic contructs comprising a BMP-4 responsive promoter operably linked to a reporter gene, with the agent, thereby producing one or more cultures of cells contacted with the agent; and
b) measuring the amount of gene product produced as a result of reporter gene expression in said one or more cultures of cells contacted with the agent and in one or more cultures of control cells;
wherein a greater or lesser amount of gene product produced as a result of reporter gene expression in said one or more cultures of cells contacted with the agent, compared to the amount of gene product in said one or more cultures of control cells is indicative that said agent reduces melanin production in cells of melanocytic lineage.

30. The method of claim 29 wherein the BMP-4 responsive promoter is the tyrosinase promoter, and a lesser amount of gene product produced as a result of reporter gene expression in said one or more cultures of cells contacted with the agent, compared to the amount of gene product in said one or more cultures of control cells, is indicative that said agent reduces melanin production in cells of melanocytic lineage.

31. The method of claim 29 wherein the BMP-4 responsive promoter is the microphthalmia-associated transcription factor promoter, and a lesser amount of gene product produced as a result of reporter gene expression in said one or more cultures of cells contacted with the agent, compared to the amount of gene product in said one or more cultures of control cells, is indicative that said agent reduces melanin production in cells of melanocytic lineage.

32. A method of identifying an agent that reduces melanin production in cells of melanocytic lineage, said method comprising:
a) transfecting cells, wherein the cells have in their cell membranes functional BMP receptor type 1A, BMP receptor type 1B and BMP receptor type 2, with a vector comprising a BMP-4 responsive promoter operably linked to a reporter gene, thereby producing transfected cells;
b) contacting the transfected cells with the agent, thereby producing one or more cultures of transfected cells contacted with the agent; and
c) measuring the amount of gene product produced as a result of reporter gene expression in said cultures of transfected cells contacted with the agent and in one or more cultures of control cells;
wherein a greater or lesser amount of gene product produced as a result of reporter gene expression in said one or more cultures of transfected cells contacted with the agent, compared to the amount of gene product in said one or more cultures of control cells, is indicative that said agent reduces melanin production in cells of melanocytic lineage.

33. The method of claim 32 wherein the BMP-4 responsive promoter is the tyrosinase promoter, and a lesser amount of gene product produced as a result of reporter gene expression in said one or more cultures of transfected cells contacted with the agent compared to the amount of gene product in said one or more cultures of control cells is indicative that said agent reduces melanin production in cells of melanocytic lineage.

34. The method of claim 32 wherein the BMP-4 responsive promoter is the microphthalmia-associated transcription factor promoter, and a lesser amount of gene product produced as a result of reporter gene expression in said one or more cultures of transfected cells contacted with the agent compared to the amount of gene product in said one or more cultures of control cells is indicative that said agent reduces melanin production in cells of melanocytic lineage.


SEQUENCE LISTING (7 sequences)

SEQ ID NO: 1 - DNA, 656 bp, Homo sapiens
tagactgttgagtacaacacgtgtaggccagaggagacagtggcctatacttgggacaaataaagaggtctgtcctatttaagaaaatcaaccctgtaaaggaaattaataggactaagtacattttagtaaggcctctaagcaggctctaaagattatgaaaaatacacgggacagcagacacaaaagcccttaaagagcatgaagactttctaagttatttcactggaagcctgatagtggggcaagtgtaaggcaaaattcttaattaaattgaaaatgataagttgaattctgtcttcgagaacatagaaaagaattatgaaatgccaacatgtggttacaagtaatgcagacccaaggctccccagggacaagaagtcttgtgttaactctttgtggctctgaaagaaagagagagagaaaagattaagcctccttgtggagatcatgtgatgacttcctgattccagccagagcgagcatttccatggaaacttctcttcctcttcacccacacactgctccatgtacctgcaaagcctgttctgtctcaaaaaagttgtttggatgagccgtgacttttttttttcttaaataatgagacaaactccagaaaaagagaaaaaagcagagcagctgacattccggcatca

SEQ ID NO: 2 - DNA, 21 bp, Artificial Sequence
  PCR primer
ggacattgctttgccatcata

SEQ ID NO: 3 - DNA, 21 bp, Artificial Sequence
  PCR primer
cagacccactaccagaacttt

SEQ ID NO: 4 - DNA, 21 bp, Artificial Sequence
  PCR primer
gttgtaaatgccaccaccatt

SEQ ID NO: 5 - DNA, 21 bp, Artificial Sequence
  PCR primer
gtctggtttcttgtcttttat

SEQ ID NO: 6 - DNA, 21 bp, Artificial Sequence
  PCR primer
tggctgaacttatgatgattt

SEQ ID NO: 7 - DNA, 21 bp, Artificial Sequence
  PCR primer
tgttggtggagaggctggtga